(19) United States
(12) Patent Application Publication
(10) Pub. No.: US 2012/0029045 A1
(43) Pub. Date: Feb. 2, 2012

(54) METHOD FOR TREATING PORT WINE STAINS

(75) Inventors: Jining Tao, Shanghai (CN); Wenhui Chen, Shanghai (CN); Yong Su, Shanghai (CN)

(73) Assignee: SHANGHAI FUDAN-ZHANGJIANG BIO-PHARMACEUTICAL CO., LTD.

(21) Appl. No.: 12/848,755

(22) Filed: Aug. 2, 2010

(51) Int. Cl. A61K 31/409; A61P 17/00

(52) U.S. Cl. 514/410

(57) ABSTRACT

A method of using photodynamic therapy to perform selective targeted therapy of biological tissue. The method includes intravenously injecting a porphyrin-based photosensitizing drug followed by irradiating the tissue with light while the drug is being injected. The duration of the irradiation and other parameters are controlled so that the selected biological tissue is treated and non-selected tissue is not damaged. By controlling the flow rate of the injection and other parameters, so that irradiation of the effected tissue overlaps with injection of drug, the target tissue is effectively treated without damage to non-target tissue.

### FIELD OF THE INVENTION

This disclosure relates to methods for treating skin disorders. Particularly, the disclosure relates to methods for treating vascular proliferation in skin tissue. More particularly, the disclosure relates to methods for treating or preventing port wine stains by using photodynamic therapy.

### BACKGROUND OF THE INVENTION

### Port Wine Stains

Port-wine stains (Nevus flammeus, PWS) are congenital birthmarks, which range in color from pale pink to dark purple. These congenital malformations of dermal capillaries are characterized by the presence of dilated capillaries in the papillary layer of the dermis. Histopathological studies of PWS show a normal epidermis overlying an abnormal plexus of dilated blood vessels located on a layer in the upper dermis with a thickness of about 0.06 mm.

PWS are usually flat, smooth and irregular in appearance, and tend to fade when they are pressed. PWS are a form of lesion which most often occurs on the face, neck and scalp, but can appear anywhere on the body, e.g., the limbs, pro-thorax and palm or back of the hand. In adulthood, thickening of the PWS lesion or the development of small lumps may occur. The area of skin affected grows in proportion to general growth and PWS is often associated with other symptoms including thickened lips or thickening of one side of the face. Other symptoms of port-wine stains include the Klippel-Trenaunay syndrome in which one limb is longer and larger than the other limb, and Sturge-Weber syndrome the symptoms of which include glaucoma and seizures.

Past treatments for PWS have included surgery to physically excise the PWS, ionizing radiation, skin grafting, magnetotherapy, cryosurgery, pharmacotherapy, electrotherapy and laser therapy (e.g. CO₂, YAG, Ar ion, copper vapor and KTP (potassium titanyl phosphate)). These methods result in undesired scarring and/or incomplete elimination of lesions.

Recently a pulse dye laser has been used to treat PWS; however, such treatment does not result in effective removal of the PWS and still results in post-treatment scarring.

Vascular-targeted photodynamic therapy (PDT) is another recent alternative approach in the treatment of PWS. In this method hematoporphyrin derivative (HpD) or photosynthesizing drug (PSD-007) is injected intravenously at a dose of 0.8-5.0 mg/kg body weight followed by argon ion laser irradiation with a wavelength range of 488.0˜514.0 nm using a power density 50˜100 mW/cm² and an energy density of 0-540 J/cm² for the light source, (Ying Gu, et al., Chinese J Laser Med Surg, 1992, 11(1): 6-10). Irradiation was applied 15-60 minutes after completion of the drug injection. This method resulted in the color of the PWS completely fading in 15% of the treated patients, and the color of the PWS partly fading in 67.5% of the treated patients. This method has a relatively long waiting duration between injection and irradiation, in which the drug may leak from blood vessels into normal epidermis and serious adverse effects like scars may occur. Another method involves applying HpD or PSD-007 at a dose of 2.5-5.0 mg/kg body weight, which is then irradiated by yttrium-aluminum-garnet (YAG) laser with a wavelength range of 488.0˜514.0 nm using a power density 50˜150 mW/cm² and energy density 90-480 J/cm² for the light source, (Chen Shurui, et. al. (Journal of Medical Aesthetics and Cosmetology, 1994, 1(2-3): 126-127).

Various photosensitizing drugs and their effective dosage previously used to treat PWS are shown in Table 1; the entireties of the listed references are incorporated herein by reference.

TABLE 1
photosensitizing drugs and their dosage in PDT for PWS

| Researchers | Prior reference | Photosensitizing drugs | Dosage | |
|---|---|---|---|---|
| Ying Gu, | Chinese J Laser Med Surg, | HpD injection, produced by Institute | 0.8~5.0 | mg/kg |
| et al. | 1992, 11(1): 6-10 | of Beijing pharmaceutical Industry | | |
| | | PsD-007 injection, produced by the | | |
| | | cond Military Medical University | | |
| Shurui Chen, | Journal of Medical | HpD injection, produced by Institute | 2.5~5.0 | mg/kg |
| et al | Aesthetics and Cosmetology, | of Beijing pharmaceutical Industry | | |
| | 1994, 1(2-3): 126-127 | PsD-007 injection, produced by the | | |
| | | cond Military Medical University | | |
| Lianxing Wang, | Laser Medicine, 1995, 5 | PsD-007 injection, produced by the | 5.0~10.0 | mg/kg |
| et al | (3): 135-137 | cond Military Medical University | | |
| Ying Gu, | Chinese J Laser Med | HMME | 4.5~6.0 | mg/kg |
| et al | Surg, 1996(15), 4: 201-204 | | | |
| Heqing Li, | Journal of Nursing Science, | HMME | 3.5~5 | mg/kg |
| et al | 1998, 1513 (5): 290-291 | | | |

Various parameters of light sources previously used are shown in Table 2.

| Researchers | Prior reference | Light intensity | | Light duration | | Energy density | |
|---|---|---|---|---|---|---|---|
| Ying Gu, | Chinese J Laser Med | 50~100 | mW/cm² | | | 90~540 | J/cm² |
| et al. | Surg, 1992, 11(1): | | | | | | |
| | 6-10 | | | | | | |
| Shurui Chen, | Journal of Medical | 50~150 | mW/cm² | | | 90~480 | J/cm² |
| et al | Aesthetics and | | | | | | |
| | Cosmetology, | | | | | | |
| | 1994, 1(2-3): 126-127 | | | | | | |
| Lianxing Wang, | Laser | 300~800 | mW/cm² | 30 min~1 | h | 360~640 | J/cm² |
| et al | Medicine, 1995, 5 | | | | | | |
| | (3): 135-137 | | | | | | |
| Ying Gu, | Chinese J Laser Med | 80~100 | mW/cm² | 35~60 | min | 168~360 | J/cm² |
| et al | Surg, 1996(15), 4: | | | | | | |
| | 201-204 | | | | | | |
| Heqing Li, | Journal of Nursing | 70~100 | mW/cm² | 30~40 | min | 150~360 | J/cm² |
| et al | Science, 1998, 1513 | | | | | | |
| | (5): 290-291 | | | | | | |

With most of these prior art laser treatments used for may PWS lesions, the threshold for epidermal damage following laser therapy is very close to the threshold for permanent blanching of the PWS.

As such, it is desirable to apply a safe and effective photodynamic therapy of PWS that allows treatment of selected layers of tissue without nonspecific damage to non-selected layers. Thus, a methodology is needed which can be effectively used to produce a high concentration difference of photosensitizing drugs between selected and non-selected tissue layers; specifically a selective photodynamic therapy that has high photosensitizing drug concentration in dilated capillaries in the papillary layer of the dermis (the selected layers), and low photosensitizing drug concentration in normal epidermis and reticular layer of the dermis (the non-selected layers). However, the plasma concentration of photosensitizing drugs sharply reaches a peak immediately after traditional intravenous injection is started and then drops rapidly. This drop in photosensitizing drug concentration in the plasma is due to most of the photosensitizing drug leaking from blood vessels into other nearby tissues. Thus, there is hardly any difference in drug concentration between the selected and non-selected layers.

Therefore, there is a need for an effective method of treatment which uniformly provides positive results, namely eliminating the abnormal color of PWS. Furthermore, such an effective treatment would prolong the therapeutic time window—the time period in which there is a photosensitizing drug concentration difference between the selected layers and the non-selected layers, so that damage to the non-selected tissue layers is prevented.

### SUMMARY OF THE INVENTION

An aspect of the present invention is a method for using photodynamic therapy (PDT) to perform selective targeted therapy of port wine stains (PWS). In certain embodiments, the method comprises the steps of:

a) injecting intravenously porphyrin-based photosensitizing drugs at a dose of 2.0-5.0 mg/kg body weight at a constant rate, with the total injection duration being 20 minutes;

b) irradiating the port wine stain lesion within 0-10 minutes after the start of the injection, with a light having a wavelength in the range of between about 400 and about 580 nm and a power density in the range of 60-100 mW/cm²; and

c) irradiating the port wine stain lesion for about 20-30 minutes, to provide an overlap between drug injection and light irradiation of about 10-20 minutes. As a result, the lesions may be effectively laser treated without damage to normal biological tissues.

Another aspect of this invention is a method of using multiple irradiating light sources to irradiate different port wine stains or different portions of a large port wine stain.

Another aspect of the invention is a method of minimizing scarring associated with the treatment of port wine stains in a patient. The method includes intravenously administering a porphyrin-based photosensitizing drug in the patient in an amount of about 2.0-5.0 mg/kg body weight for about 20 minutes with a constant flow rate. In addition the method includes a step of irradiating a portion of biological tissue having a port wine stain with irradiating light within 0-10 minutes after the start of injection for about 20-30 minutes so that the overlap between the intravenous administering step and the irradiating step is approximately 10-20 minutes.

Another aspect of the invention is a method of treating multiple port wine stains in a patient. The method includes intravenously injecting in the patient, a porphyrin-based photosensitizing drug in an amount of about 2.0-5.0 mg/kg body weight for about 20 minutes with a constant flow rate. The method also includes a step of irradiating a first port wine stain with irradiating light within 0-10 minutes after the start of injection for about 20-30 minutes, so that the overlap between the intravenous drug injection of step and light irradiation is approximately 10-20 minutes. The irradiating light has a wavelength in the range of between about 480 and about 580 nm and a power density in the range of about 60-100 mW/cm². Treatment of a second port wine stain is started about is 2 to about 4 weeks after the treatment of the first port wine stain. The steps of treating the second port wine stain can be substantially the same as for the treatment of the first port wine stain

### BRIEF DESCRIPTION OF THE DRAWINGS

FIG. 1 shows the dynamic change of the concentration of HpD in chicken blood over post-injection time.

FIG. 2 shows the dynamic change of the concentration of HpD and HMME in chicken blood over post-injection time.

FIG. 3 shows the plasma drug concentration-time curves of HMME after intravenous injection over post-injection time.

### DETAILED DESCRIPTION OF THE PREFERRED EMBODIMENTS

The inventors have studied pharmacokinetics of porphyrin-based photosensitizing drugs in test animals and in humans and studied photo damage to selected tissues and non-selected tissues at various times after administration of these drugs in order to find a safe and effective treating period. In particular, the inventors have discovered therapeutic parameters, e.g., the dosage of photosensitizing drugs, the administration route, the light dose, the start-time and the end-time of the irradiation that provide an improved treatment for the reduction of port wine stains.

It has been surprisingly discovered that by performing irradiation for a period of time overlapping intravenous injection of the photosensitizing drug, improved therapeutic results are obtained.

### Photodynamic Therapy (PDT)

PDT treatment according to the invention involves three key components: a photosensitizing drug, light, and tissue oxygen. The photosensitizing drug used in the inventive method is a poryphin based drug that can be administrated systematically and can aggregate at the PWS lesions and be excited by light of a specific wavelength. The photosensitizing drug used in the inventive method may be excited from a ground singlet state to an excited singlet state. After excitation, the photosynthesizing drug undergoes a reaction with other compounds in the tissue to form singlet oxygen and/or other radicals. Ultimately, these destructive reactions kill lesion cells through direct cytotoxicity and vascular closure.

### Mechanism of PDT for Port-Wine Stains

A possible mechanism of PDT for PWS is summarized as follows: while shining the light at the PWS lesion during or shortly after intravenous injection of the photosensitizing drug, i.e., while the drug is still highly concentrated inside the circulation (including within the PWS vessels), the drug distributes mainly to endothelial cells and diffuses little to the surrounding epithelium tissue. As such, the photochemically induced reactive oxygen species (ROS) may be located within the vessels and, therefore, selectively effectively treat the endothelial cells without harming the normal epidermis, which is free of photosensitizing drugs. The normal dermal tissue beneath the lesion is protected from the laser damage because of the shallow penetration of the laser that is used. PDT destruction of PWS vessels leads to color blanching of the PWS lesion. Since the amount of photosensitizing drug outside the vessels is low, the vascular acting PDT-induced skin lesion is negligible or reversible.

### PDT Procedure

In general, PDT procedures involve the combination of photosensitizing drugs and light sources. The PDT procedures according to some embodiments of the invention minimize scarring associated with the treatment of port wine stains in a patient. The varied parameters of photosensitizing drugs may include dosage and means of delivery, and the parameters of the light source may include light flux (light dosage, light energy density), light intensity and light duration. Each parameter in effective and selective PDT is interrelated so an optimal combination is controlled for selective treatment of the lesions. The present invention provides a specific set of such parameters providing for an enhanced selected treatment of PWS lesions.

Since edema is the most common side effect occurring in PDT for PWS, it is reduced by treatment with antihydropic agents such as prednisone. The lesions may form a scar or crust 1 week after PDT and may last for 2-4 weeks. As used herein, the term scar means a mark left on the skin by the healing of injured tissue and the term crust means dried serum, pus or blood mixed with epithelial and/or bacterial debris. Any resulting infection is treated after PDT by the administration of anti-infective drugs, such as antibiotics.

Therapeutic effect of treatment is assessed by examining the color of lesions following treatment, as set forth in Table 3, below.

The effectiveness of PWS treatment with PCT is categorized as being Excellent (I), Good (II), Effective (III) or Ineffective (IV). As shown in Table 3, an Excellent grade means that the color of the PWS completely fades (i.e., >90%).

TABLE 3
Therapeutic grade of PWS treatment with PDT

| Therapeutic grade | Clinical situation |
|---|---|
| I (excellent) | color totally fades (≧90%) |
| II (good) | color almost completely fades (≧60%, <90%) |
| III (effective) | color partly fades (≧20%, <60%) |
| IV (ineffective) | color does not fade (<20%) |

In some embodiments of the present invention, the porphyrin-based photosensitizing drugs may comprise a hematoporphyrin derivative (HpD), photosynthesizing drug (PsD-007), hematoporphyrin monomethyl ether (HMME), e.g., (HEMOPORFIN) or porfimer sodium, e.g., (PHOTOFRIN). In certain embodiments, the porphyrin-based photosensitizing drug is HEMOPORFIN.

In some embodiments, the treatment method includes irradiation of the lesion 0-10 minutes after the start of injection of the porphyrin based drug. The irradiating light has a wavelength in the range of between about 200 and about 700 nm and a power density in the range of 10-500 mW/cm². In certain embodiments the irradiation wavelength is in the range between about 400 and about 580 nm and the power density is about 60-100 mW/cm².

The irradiating light source used in the treatment methods of the invention maybe a continuous laser or quasi-continuous laser. As used herein, a continuous laser is a laser which transmits light continuously and a quasi-continuous laser is a laser which is switched on for certain time intervals during treatment. In certain embodiments, the irradiating light source is selected from an Ar-ion laser (514.5 nm and 488.0 nm), a KYP laser (pulsed Nd:YAG laser, 532 nm), and copper vapor laser (510.6 nm and 578.2 nm) and light emitting diode (LED). In certain embodiments, the laser is a KYP laser (pulsed Nd: YAG laser, 532 nm). In some embodiments, particularly for treating adults, the power density applied to the lesion may be about 80 to about 100 mW/cm², while in children it may be about 60 to about 80 mW/cm². As used herein an adult is a person who is fully grown or developed past the stage of puberty and a child is a person between the stages of birth and puberty. Depending on the size of the patient being treated, it may be preferable to adapt the treatment for a child or adult accordingly. The skilled practitioner will be able to make such adjustments in treatment.

In some embodiments, particularly for patients having a large lesion, multiple laser spots (e.g., double laser spots) may be applied so that by combining multiple laser spots, the entire portion of the large lesion is covered with irradiating light. In certain embodiments of the invention, treatment of PWS over multiple treatment applications is used, the interval of each two adjacent treatments depending on the t_{1/2} of the photosensitizing drugs used.

If the same portion of the lesion requires more than one treatment, the interval of time between two adjacent treatments may be at least 2 months to 4 months. If a different portion of the lesion needs treatment after the first treatment, the interval of time between two adjacent treatments may be at least 2 weeks to 4 weeks.

There is no maximum amount of time between treatments, but the interval between treatments is governed by whether the same portion of a lesion is being treated multiple times.

The PWS in the dermis is irradiated through the epidermis for a time period sufficient to selectively destroy cutaneous blood vessels within the PWS. As a result, the port wine stain is destroyed without substantial biological damage to the epidermis.

### Photosensitizing Drug and its Dosage

The photosensitizing drugs used in the present methods are porphyrin-based photosensitizing drugs which can be produced by any method, such as the methods described by Daming Qin (Journal of Biology, 1991, 42(4): 4-6), Deyu Xu (Chinese Journal of Pharmaceuticals, 1989, 20(10): 440-446), Wenhui Chen (Chinese J Laser Med Surg, 1993, 12(1): 3-7) or the method in CN01131939, and Porfimer (PHOTOFRIN) provided by Quadra Logic Technologies Phototherapeutics Inc. The contents of which are incorporated by reference.

The following examples illustrate methods and effects of PDT for PWS. The examples are in no way intended to limit the scope of the invention. The test conditions not described here are common conditions to skilled persons, or they are the conditions advised by the manufacturers.

### EXAMPLES

### Example 1

### Pharmacokinetics Studies

### Dynamic Change of the Concentration of Poryphin Drugs in Chicken Blood

### HpD and PsD-007

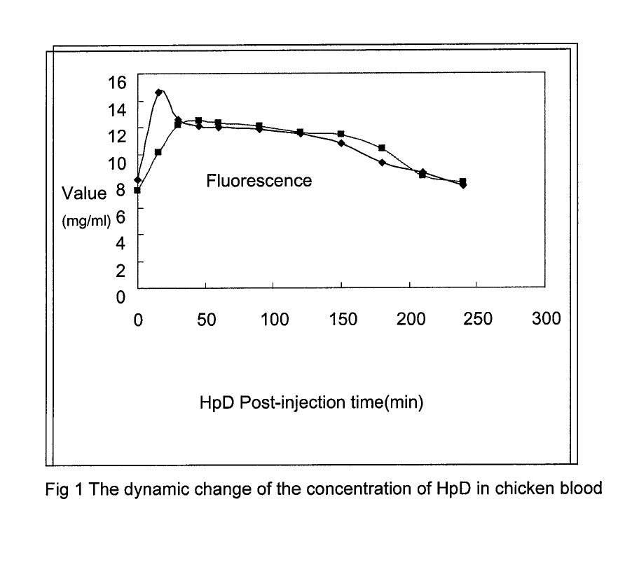

The concentrations of HpD and PsD-007 in a test animal's (i.e., chicken) blood was monitored by injecting the drug into the neck vein at a dose of 10 mg/kg of body weight, and taking blood samples approximately every 10 minutes. The results are shown in Table 4 and FIG. 1.

TABLE 4
Dynamic change of the concentration
of HpD and PsD-007 in chicken blood

Fluorescence value (mg/ml)

| Post-injection time (min) | HpD | PsD-007 |
|---|---|---|
| 0 | 8.1 | 7.3 |
| 15 | 14.6 | 10.1 |
| 30 | 12.6 | 12.2 |
| 45 | 12.1 | 12.5 |
| 60 | 12.0 | 12.3 |
| 90 | 11.8 | 12.1 |
| 120 | 11.5 | 11.6 |
| 150 | 10.8 | 11.4 |
| 180 | 9.3 | 10.4 |
| 210 | 8.6 | 8.3 |
| 240 | 7.6 | 7.8 |

### HpD and HMME

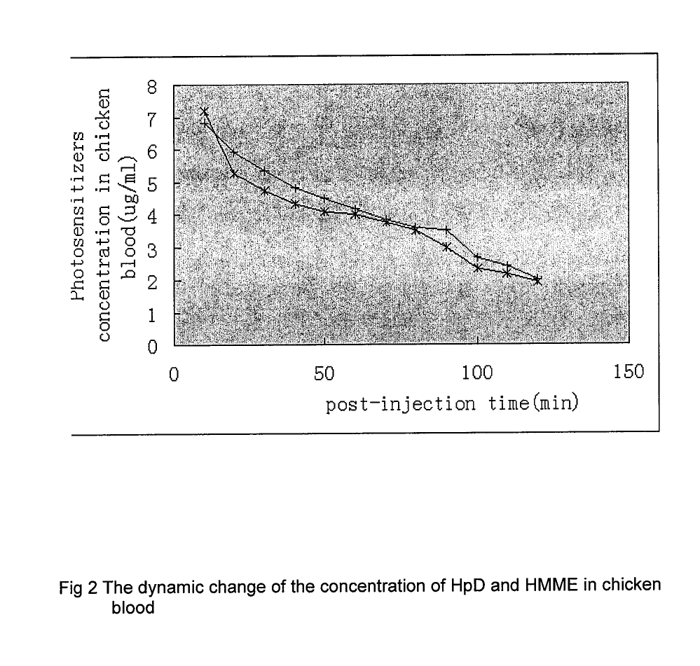

HpD and HMME in chicken blood were monitored at various times after injection. HpD or HMME was injected in chicken neck veins at a dose of 10 mg/kg, taking a blood sample every 10 minutes following injection. The results shown in Table 5 and FIG. 2 show that serum concentrations of both drugs reach a peak 10 minutes after injection and then drop. The two curves shown in FIG. 2 have generally the same pattern.

TABLE 5
Dynamic change of the concentration
of HpD and HMME in chicken blood

Photosensitizer concentration (mg/ml)

| post-injection time(min) | HpD | HMME |
|---|---|---|
| 10 | 7.17 | 6.83 |
| 20 | 5.25 | 5.92 |
| 30 | 4.75 | 5.33 |
| 40 | 4.33 | 4.83 |
| 50 | 4.08 | 4.49 |
| 60 | 3.99 | 4.17 |
| 70 | 3.75 | 3.83 |
| 80 | 3.5 | 3.58 |
| 90 | 2.99 | 3.51 |
| 100 | 2.33 | 2.67 |
| 110 | 2.17 | 2.42 |
| 120 | 1.91 | 1.99 |

### Example 2

### Pharmacokinetics of HMME in Human Body

Porphyrin-based photosensitizing drugs were injected intravenously at a dose of 2.5 and 5.0 mg/kg body weight for 20 minutes with a constant flow rate, and the pharmokinetics measured. Blood samples were taken at 5, 10, 20, 25, 30, 40, 50, 80, 110, 140, 200, 260 and 380 minutes separately to measure the serum concentrations of porphyrin-based photosensitizing drugs.

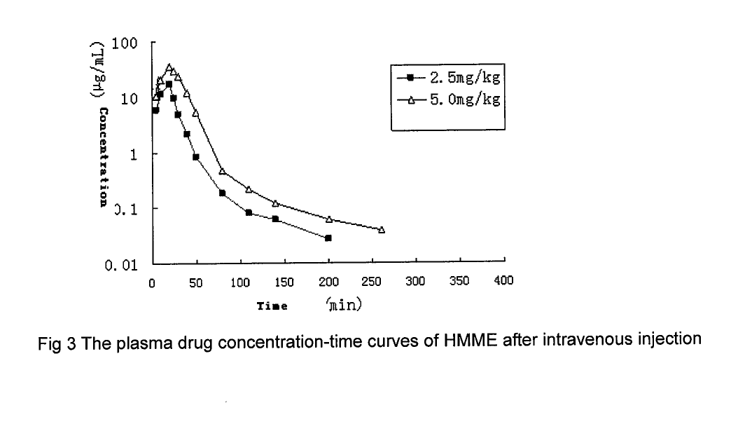

The pharmacokinetic results shown in FIG. 3 show that the Cₘₐₓ values of each dose is 17.491±7.045 and 35.724±4.539 μg·mL⁻¹ respectively, the AUC_{0˜n} value is 6.342±2.824 and 17.531±3.467 μg·mL⁻¹·h, respectively, and the t_{1/2} values are 1.26±0.33 and 1.31±0.33 h, respectively.

### Example 3

### Therapeutic Method

### Assessment on PTD for PWS

A solution of 1 ml saline was injected in the superficial vein (e.g. median cubital vein) of patients to ensure no liquid leaked into tissues adjacent to the blood vessels into which the injection was made. At the same site, HMME was injected intravenously for 20 minutes with a constant flow rate by using an infusion pump. The doses applied were 2.5 mg/kg body weight or 5.0 mg/kg body weight. Next, 2-4 ml of saline solution was again injected to prevent the drugs from aggregating locally. Irradiation with KTP532 laser was then applied to the patient's lesion site 0-10 minutes after the start of injection, for a total duration of irradiation of either 20 minutes (denoted as the 20 min group), or 30 minutes (denoted as the 30 minute group). The laser had a wavelength in the range of between about 532 nm and a power density of about 80-100 mW/cm².

When irradiation was started immediately after the start of injection, the overlap between the injection and irradiation was about 20 minutes, when irradiation started 5 minutes after the start of injection, the overlap between drug injection and light irradiation was approximately 15 minutes, and when irradiation was started 10 minutes after the start of injection, the overlap was about 10 minutes. The therapeutic results were measured 8 weeks after the treatment.

### Example 4

### Test Results

### Part A

A dose of 5.0 mg/kg body weight of the poryphin based drug was injected, total irradiation durations were 20 min and 30 min respectively, and the effects and adverse effects were assessed.

### Effect Assessment

The results as shown in Table 6 show that in the 20 min group, the excellence rate was 10.0%, significant response rate was 55.0% and response rate was 80.0%. In the 30 min group, the excellence rate was 36.8%, significant response rate was 78.9% and response rate was 94.7%.

TABLE 6
Systematic effect assessment (8 weeks after treatment)

| | | | | | | | significant | |
|---|---|---|---|---|---|---|---|---|
| | | | | | | Excellent | response | response |
| | N | Excellent | good | Moderate | ineffective | rate % | rate % | rate % |
| 20 min | 20 | 2 | 9 | 5 | 4 | 10.0 | 55.0 | 80.0 |
| group | | | | | | | | |
| 30 min | 19 | 7 | 8 | 3 | 1 | 36.8 | 78.9 | 94.7 |
| group | | | | | | | | |

ps:
Excellent rate = Excellent/total cases × 100%,
significant response rate = (excellent + good)/total cases × 100%,
response rate = (excellent + good + moderate)/total cases × 100%.

The effect on different types of PWS, was also assessed as shown in Table 7, which shows that the excellence rate and significant response rate on red type lesions was higher than purple type lesions for both the 20 min group and the 30 min group.

TABLE 7

| | | | | | | | |
|---|---|---|---|---|---|---|---|
| | | | | | | Significant | |
| | | | | | Excellent | response | Response |
| N | Excellent | Good | Moderate | Ineffective | rate % | rate % | rate % |

20 min group

| | | | | | | | | |
|---|---|---|---|---|---|---|---|---|
| Red | 1 | 2 | 7 | 2 | 0 | 2/11 | 9/11 | 11/11 |
| | 1 | | | | | | | |
| Purple | 6 | 0 | 2 | 3 | 1 | 0/0 | 2/6 | 5/6 |
| Thickening | 3 | 0 | 0 | 0 | 3 | 0/0 | 0/0 | 0/0 |

30 min group

| | | | | | | | | |
|---|---|---|---|---|---|---|---|---|
| Red | 1 | 6 | 2 | 1 | 1 | 6/10 | 8/10 | 9/10 |
| | 0 | | | | | | | |
| Purple | 8 | 1 | 5 | 2 | 0 | 1/8 | 6/8 | 8/8 |
| Thickening | 1 | 0 | 1 | 0 | 0 | 0/0 | 1/1 | 1/1 |

ps:
Excellent rate = Excellent/total cases × 100%,
significant response rate = (excellent + good)/total cases × 100%,
response rate = (excellent + good + moderate)/total cases × 100%

### Adverse Effects

The most common adverse temporary effects included swelling, burning, redness, pain, blister and crust formation in PDT for PWS and these symptoms occurred in almost every case. Scar or crust thickness was measured as thickening of crusts. The crust levels served as an assessment index for scars.

The adverse effects in the subjects in the present method were mild or moderate; the subjects in the present study reported feeling “nothing at all.” However the 30 min treatment group showed worse adverse effects compared to the 20 min treatment group as shown in Table 8. No serious-thick crusts occurred and no scars were present in any patient treated for 20 min. (Table 9), which indicates that the present invention unexpectedly reduces and in some cases, eliminates scar formation after treatment.

TABLE 8
the level of adverse effects

| | 20 min group | | | | 30 min group | | | |
|---|---|---|---|---|---|---|---|---|
| Adverse | | Mode- | | Rate | | Mode- | | Rate |
| effect | Mild | rate | total | (%) | Mild | rate | total | (%) |
| Swelling | 9 | 9 | 18 | 90.0 | 6 | 12 | 18 | 90.0 |
| Burning | 0 | 0 | 0 | 0.0 | 0 | 1 | 1 | 5.0 |
| Redness | 0 | 0 | 0 | 0.0 | 0 | 2 | 2 | 10.0 |
| Pain | 1 | 9 | 10 | 50.0 | 3 | 12 | 15 | 75.0 |
| Blister | 0 | 0 | 10 | 0.0 | 2 | 0 | 2 | 10.0 |

TABLE 9
crust levels

| | |
|---|---|
| 20 min group | 30 min group |
| N (%) | N (%) |

| | | | | |
|---|---|---|---|---|
| None | 5 | (25.0) | 3 | (15.0) |
| Thin and fractional | 10 | (50.0) | 5 | (25.0) |
| Thin and full | 4 | (20.0) | 8 | (40.0) |
| Moderate thick and fractional | 1 | (5.0) | 1 | (5.0) |
| Moderate thick and full | 0 | (0.0) | 3 | (15.0) |
| Total | 20 | | 20 | |

Chi-square test, P = 0.165

### Part B

In this example doses of 2.5 and 5.0 mg/kg body weight respectively, were applied, the patient was irradiated for 20 min, and the therapeutic effects and the adverse effects were assessed.

### Effect Assessment

The results in Table 10 show that in the 5.0 mg/kg group, excellent rate was 5.0%, significant response rate was 40.0% and response rate was 75.0%; while in 2.5 mg/kg group, the excellent rate was 0%, significant response rate was 2.5% and response rate was 40%.

TABLE 10
Systematic effect assessment (8 weeks after treatment)

| | | | | | | | significant | response |
|---|---|---|---|---|---|---|---|---|
| | | | | | | Excellent | response | rate |
| | N | Excellent | Good | Moderate | Ineffective | rate % | rate % | % |
| 5.0 mg/kg | 40 | 2 | 14 | 14 | 10 | 5.0 | 40.0 | 75.0 |
| group | | | | | | | | |
| 2.5 mg/kg | 40 | 0 | 1 | 15 | 24 | 0.0 | 2.5 | 40.0 |
| group | | | | | | | | |
| Placebo | 20 | 0 | 0 | 3 | 17 | 0.0 | 0.0 | 15.0 |
| group | | | | | | | | |

ps:
Excellent rate = Excellent/total cases × 100%,
significant response rate = (excellent + good)/total cases × 100%,
response rate = (excellent + good + moderate)/total cases × 100%

The effect on different types of PWS was also assessed, as shown in Table 11: the excellent rate and significant response rate for red type lesions was higher than purple lesions for both the 5.0 mg/kg group and 2.5 mg/kg group. The 5.0 mg/kg group responded better than the 2.5 mg/kg group.

TABLE 11
Systematic effect assessment for various types of PWS

| | | | | | | | | |
|---|---|---|---|---|---|---|---|---|
| | | | | | | Significant | | |
| | | | | | Excellent | response | Response | |
| N | Excellent | Good | Moderate | Ineffective | rate % | rate % | rate % | N |

5.0 mg/kg group

| | | | | | | | | |
|---|---|---|---|---|---|---|---|---|
| Red | 14 | 2 | 7 | 4 | 1 | 14.3 | 64.3 | 92.9 |
| Purple | 24 | 0 | 7 | 9 | 8 | 0.0 | 29.2 | 66.7 |
| Thickening | 2 | 0 | 0 | 1 | 1 | 0/2 | 0/2 | 1/2 |

2.5 mg/kg group

| | | | | | | | | |
|---|---|---|---|---|---|---|---|---|
| Red | 16 | 0 | 1 | 8 | 7 | 0.0 | 6.3 | 56.3 |
| Purple | 17 | 0 | 0 | 3 | 14 | 0.0 | 0.0 | 17.6 |
| Thickening | 7 | 0 | 0 | 4 | 3 | 0/7 | 0/7 | 4/7 |

Placebo group

| | | | | | | | | |
|---|---|---|---|---|---|---|---|---|
| Red | 12 | 0 | 0 | 2 | 10 | 0.0 | 0.0 | 16.7 |
| Purple | 6 | 0 | 0 | 0 | 6 | 0/6 | 0/6 | 0/6 |
| Thickening | 2 | 0 | 0 | 1 | 1 | 0/2 | 0/2 | 1/2 |

ps:
Excellent rate = Excellent/total cases × 100%,
significant response rate = (excellent + good)/total cases × 100%,
response rate = (excellent + good + moderate)/total cases × 100%

### Adverse Effects

No serious-thick crusts occurred and no scars were formed in any treated patients as shown in Table 12. This result demonstrates that the present invention reduces and in some cases, eliminates this discomfort and scarring.

TABLE 12
Crust levels

| | | Mild | | Moderate | | Serious | | Total | |
|---|---|---|---|---|---|---|---|---|---|
| Group | N | N | Rate (%) | N | Rate (%) | N | Rate (%) | N | Rate (%) |
| 5.0 mg/kg | 50 | 30 | 60.0 | 14 | 28.0 | 0 | 0.0 | 44 | 88.01 |
| group | | | | | | | | | |
| 2.5 mg/kg | 49 | 17 | 34.7 | 0 | 0.0 | 0 | 0.0 | 17 | 34.7 |
| group | | | | | | | | | |
| Placebo | 20 | 0 | 0.0 | 0 | 0.0 | 0 | 0.0 | 0 | 0.0 |
| group | | | | | | | | | |

Although specific embodiments of the invention have been described and illustrated, it is to be understood that modifications can be made without departing from the invention's sprit and scope. The scope of the invention as defined in the appended claims is intended to cover these and other variation.

What is claimed is:

1. A method for treating biological tissue in a patient in need thereof, comprising the steps of:
intravenously injecting in the patient, a porphyrin-based photosensitizing drug in an amount of about 2.0-5.0 mg/kg body weight for about 20 minutes with a constant flow rate; and
irradiating a portion of the biological tissue with irradiating light from an irradiating light source within 0-10 minutes after the start of injection for about 20-30 minutes;
wherein:
the irradiating light has a wavelength in the range of between about 480 and about 580 nm and a power density in the range of about 60-100 mW/cm²; and
the overlap between the intravenous drug injection step and light irradiation step is approximately 10-20 minutes.

2. The method of claim 1, wherein the at least one portion of the biological tissue is a red port wine stain.

3. The method of claim 1, wherein said porphyrin-based photosensitizing drug is selected from hematoporphyrin derivative (HpD), photosynthesizing drug (PsD-007), hematoporphyrin monomethyl ether (HMME) and porfimer.

4. The method of claim 1, wherein said amount of porphyrin-based photosensitizing drug is 5.0 mg/kg body weight.

5. The method of claim 1, comprising multiple irradiating light sources, wherein a first irradiating light source irradiates a first portion of the biological tissue and a second irradiating light source irradiates different portion of the biological tissue.

6. The method of claim 1, wherein the at least one irradiating light source is a continuous laser or a quasi-continuous laser.

7. The method of claim 6, wherein said laser is a 532 nm KYP laser.

8. The method of claim 6, wherein the power density of the laser is in the range of 80-100 mW/cm² and the patient is an adult.

9. The method of claim 1, wherein the total duration of the irradiating step is about 20 minutes.

10. The method of claim 1, wherein the irradiating step begins 5-10 minutes after the start of the injecting step.

11. The method of claim 1, further comprising:
repeating steps (a) and (b) after an interval of about 2 months to about 4 months.

12. A method of minimizing scarring associated with the treatment of port wine stains in a patient, comprising the steps of:
intravenously administering a porphyrin-based photosensitizing drug in the patient in an amount of about 2.0-5.0 mg/kg body weight for about 20 minutes with a constant flow rate;
irradiating a portion of biological tissue having a port wine stain with irradiating light within 0-10 minutes after the start of injection for about 20-30 minutes;
wherein the overlap between the intravenous administering step and the irradiating step is approximately 10-20 minutes.

13. The method of claim 12, wherein the total duration of the irradiating step is about 20 minutes.

14. The method of claim 12, wherein said porphyrin-based photosensitizing drug is selected from hematoporphyrin derivative (HpD), photosynthesizing drug (PsD-007), hematoporphyrin monomethyl ether (HMME) and porfimer.

15. The method of claim 12, wherein said amount of porphyrin-based photosensitizing drug is 5.0 mg/kg body weight.

16. The method of claim 12, wherein, the irradiating light is emitted from a 532 nm KYP laser having a power density in the range of 80-100 mW/cm² and the patient is an adult.

17. A method of treating multiple port wine stains in a patient comprising the steps of:
(a) intravenously injecting in the patient, a porphyrin-based photosensitizing drug in an amount of about 2.0-5.0 mg/kg body weight for about 20 minutes with a constant flow rate;
(b) irradiating a first port wine stain with irradiating light within 0-10 minutes after the start of injection for about 20-30 minutes;
wherein:
the irradiating light has a wavelength in the range of between about 480 and about 580 nm and a power density in the range of about 60-100 mW/cm², and
the overlap between step (a) and step (b) is approximately 10-20 minutes, and;
(c) repeating step (a) and irradiating a second port wine stain with irradiating light after an interval of about 2 weeks to about 4 weeks,
wherein, the second port wine stain is irradiated within 0-10 minutes after the start of
injection for about 20-30 minutes and the overlap between the intravenous drug injection of and light irradiation is approximately 10-20 minutes.

18. The method of claim 17, wherein the total duration of the irradiating step is about 20 minutes.

19. The method of claim 18, wherein said porphyrin-based photosensitizing drug is selected from hematoporphyrin derivative (HpD), photosynthesizing drug (PsD-007), hematoporphyrin monomethyl ether (HMME) and porfimer.

20. The method of claim 17, wherein, the irradiating light is emitted from a 532 nm KYP laser having a power density in the range of 80-100 mW/cm² and the patient is an adult.
